(19) United States
(12) Patent Application Publication
(10) Pub. No.: US 2024/0285481 A1
(43) Pub. Date: Aug. 29, 2024

(54) ANHYDROUS COSMETIC COMPOSITION INCLUDING OIL AND PLATY MINERAL, AND METHODS OF USE

(71) Applicant: L'OREAL, PARIS (FR)

(72) Inventor: Alexandra Nicole GOODE, Bloomfield, NJ (US)

(73) Assignee: L'OREAL, PARIS (FR)

(21) Appl. No.: 18/114,639

(22) Filed: Feb. 27, 2023

(51) Int. Cl. A61K 8/02; A61K 8/04; A61K 8/26; A61K 8/31; A61K 8/891; A61Q 1/04

(52) U.S. Cl. CPC A61K 8/0258; A61K 8/042; A61K 8/26; A61K 8/31; A61K 8/891; A61Q 1/04; A61K 2800/43

(57) ABSTRACT

Method and composition for making up the lips. Method includes applying to the lips an anhydrous composition. The composition includes at least one nonvolatile silicone oil; at least one volatile oil; at least one film-former; at least one colorant; at least one platy mineral; and a mattifying agent. The at least one nonvolatile silicone oil and the at least one volatile oil are present in a weight ratio of nonvolatile silicone oil to volatile oil from about 4:1 to about 10:1.

### FIELD OF THE INVENTION

The present invention relates to a cosmetic composition and method for making up and/or enhancing the appearance of a keratinous substrate such as lips and, in particular, for anhydrous compositions including nonvolatile silicone oil, volatile oil, film-former, colorant, and a platy mineral.

### BACKGROUND OF THE INVENTION

Cosmetic compositions used to make up or enhance the appearance of a user's skin are often required to impart various properties such as long wear, transfer resistance, mattity and comfort. The inventors of the present invention have recognized that it would be desirable to have a composition that provides certain sensory effects in conjunction with one or more of the above properties. In particular, the inventors of the present invention have recognized that it would be desirable to provide enhanced tactile properties such as comfort and/or reduced tack, yet still have optical characteristics such as mattity and optical blurring. Ideally one would also be able to achieve these properties with long wear/transfer resistance. However, the formulation of cosmetic products that can deliver these properties at the same time can pose some challenges. Typically one or more of the above motioned properties cannot easily be maintained at sufficiently desirable levels.

The inventors of the instant invention surprisingly now have discovered that using particular oils and minerals in particular arrangement provides a solution to the above-mentioned drawbacks.

### BRIEF SUMMARY OF THE INVENTION

According to one embodiment of the present invention, there is provided an anhydrous composition which is long wearing and transfer resistant, while at the same time provides superior comfort, non-tacky feel and has a unique sensory profile.

According to certain aspects, a method of making up the lips includes applying to the lips an anhydrous composition. The composition includes comprising at least one nonvolatile silicone oil; at least one volatile oil; at least one film-former; at least one colorant; at least one platy mineral; and a mattifying agent. The at least one nonvolatile silicone oil and the at least one volatile oil are present in a weight ratio of nonvolatile silicone oil to volatile oil from about 4:1 to about 10:1.

According to certain aspects the anhydrous lip cosmetic composition is a liquid lip composition and includes from about 40% by weight to about 80% by weight an oil system comprising at least one nonvolatile silicone oil and at least one volatile oil. It further includes at least one silicone film-former; at least one silicone elastomer; from about 6.5% to about 10% of one or more micas; and silica aerogel. The at least one nonvolatile silicone oil and the at least one volatile oil are present in a weight ratio of nonvolatile silicone oil to volatile oil from about 4:1 to about 10:1.

According to certain embodiments, the above method and/or composition is further characterized by one or more of the following attributes, alone or in combination: the at least one platy mineral is present in a weight ratio of platy mineral to oil system that is from about 1:10 to about 1:5; the at least one volatile oil is present in a concentration in the composition from about 4% by weight to about 10% (e.g., 8%) by weight; the volatile oil is selected from one or more C₈ to C₁₆ alkanes; the mattifying agent comprises a silica aerogel, and the silica aerogel is present in a concentration in the composition from about 0.6% by weight to about 2% by weight; the colorant is present in a concentration in the composition from about 0.6% by weight to about 3.5% by weight; the composition has less than about 4% of wax; the at least one nonvolatile silicone oil has a weighted average viscosity from about 50 cps to about 100 cps, and/or the film-former comprises a siloxysilicate resin.

### DETAILED DESCRIPTION

As used herein, the expression “at least one” means one or more and thus includes individual components as well as mixtures/combinations.

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the number. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. Unless otherwise indicated, all concentrations shown as percentages are concentrations by weight and also, unless otherwise indicated, relate to the entire composition as a whole.

“Film-former” or “film forming agent” or “film forming polymer” or “film forming resin” as used herein mean a polymer or resin that leaves a film on the substrate to which it is applied, for example, after a solvent accompanying the film-former has evaporated, absorbed into and/or dissipated on the substrate. A “silicone film-former” is a film-former that includes at least one silicone (a silicone atom bonded directly to an oxygen atom and also to a carbon atom such as in an organic moiety).

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours and retains rich color even after eating.

“Liquid” or “liquid cosmetic” or “liquid lipstick” or “liquid composition” means a composition having a fixed volume, flows to cover the bottom and assumes the shape of the portion of the container it fills and is slightly compressible (as disclosed in General chemistry, Fourth Edition 2005, p. 434. This is meant to exclude conventional stick-based lipsticks that are formed by molding and do not flow as a liquid would.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion and the one far along described.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphategroups, siloxane groups, and polysiloxane groups. The substituent(s) may be further substituted.

“Substantially free” as it is used herein means that while it is preferred that no amount of the specific component be present in the composition, it is possible to have small amounts of it in the compositions of the invention provided that these amounts do not materially affect at least one, preferably most, of the advantageous properties of the conditioning compositions of the invention. In certain embodiments, substantially free means less than about 2% of the identified ingredient, such as less than about 1%, such as less than about 0.5% based on the composition as a whole (or a particular component, if indicated). The term “anhydrous” means substantially free of water.

Numerical ranges are inclusive of endpoints and meant to include all combinations and sub-combinations. For example, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

The compositions and methods of the present invention can comprise, consist of, or consist essentially of the essential elements and limitations of the invention described herein, as well as any additional or optional ingredients, components, or limitations described herein or otherwise useful.

### Film-Former

The cosmetic compositions of the present invention include at least one film-former and the total amount of at least one film-former may be present in the composition in a concentration by weight from about 1% to about 25% such as 1%, 2%, 4% or 5% to about 10%, 15%, 20% or 25%.

Examples of film-formers include silicone resins such as those described in U.S. Pat. Nos. 5,505,937, 5,911,974, 5,965,112, 5,985,298, 6,074,654, 6,780,422, 6,908,621, the disclosures of which are hereby incorporated by references.

According to certain embodiments the composition includes a silicone resin selected from a siloxysilicate resin, a silsequioxane resin, and combinations thereof. According to certain embodiments the silicone film-former is a siloxysilicate resin. One non-limiting example of a siloxysilicate in accordance with the present invention is trimethylsiloxysilicate, which may be represented by the following formula:

[(CH3)3SiO]x(SiO4/2)y
wherein x and y may, for example, range from 50 to 80. Such siloxysilicates are commercially available from General Electric, Dow Corning, Wacker, Milliken, Siltech, Grant Industries, Momentive and Shin-Etsu Silicones under the tradename Resin MQ®.

According to another embodiment of this invention, the compositions may contain silsesquioxane resins, including comprise at least one polypropyl silsesquioxane film forming resin.

Silsesquioxane resins are a specific form of silicone resin. Silicone resin nomenclature is known in the art as “MDTQ” nomenclature, whereby a silicone resin is described according to the various monomeric siloxane units which make up the polymer. Each letter of “MDTQ” denotes a different type of unit. When the film forming resin is made up predominantly of tri-functional units (or T units), it is generally called a silsesquioxane resin, which is described, for example in US 2006/0292096, herein incorporated by reference.

Examples of silsesquioxane resins that may be used in the present invention are alkyl silsesquioxane resins that are silsesquioxane homopolymers and/or copolymers having an average siloxane unit of the general formula R1n SiO(4-n)/2, wherein each R1 is a propyl group, wherein more than 80 mole % of R1 represent a C3-C10 alkyl group, n is a value of from 1.0 to 1.4, and more than 60 mole % of the copolymer comprises R1SiO3/2 units. As each R1 is a propyl group these polymers are called polypropylsilsesquioxane resins or “t-propyl” silsesquioxane resins. These resins and methods of making them are described, for example in U.S. Pat. No. 8,586,013, 2012/0301415, 2007/0093619, and 2006/0292096, all of which are herein incorporated by reference.

A non-limiting example of a polypropylsilsesquioxane resin suitable for use in the present invention is commercially available from Dow Corning as Dow Corning 670 Fluid or Dow Corning 680 Fluid. These Dow Corning resins have a general formula of RnSiO(4-n)/2 wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises RSiO3/2 units, and having a hydroxyl or alkoxy content from 0.2 to 10% by weight, for example between 1 and 4% by weight, preferably between 5 and 10% by weight, and more preferably between 6 and 8% by weight. Preferably, the polypropylsilsesquioxane resin has a molecular weight from about 5000 to about 30,000 and a Tg from about −5° C. to about 5° C.

Another embodiment of this invention exemplifies the composition containing at least one siloxysilicate resin, at least one silsesquioxane resin and/or mixture thereof.

### Polyorganosiloxane Copolymer

Other silicone film-formers suitable for use in compositions of the present invention include polyorganosiloxane-containing polymers. The polyorganosiloxane-containing polymer useful herein is a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. Non-limiting examples of polyorganosiloxane-containing polymers are disclosed, for example in U.S. Pat. No. 8,945,525, the disclosure of which is hereby incorporated by reference.

Additional polyorganosiloxane-containing polymers which may be used in the composition of the invention include those described in documents U.S. Pat. Nos. 5,874,069, 5,919,441, 6,051,216, and 5,981,680, the entire contents of which are hereby incorporated by reference.

A preferred polyorganosiloxane-containing polymer for use in the present invention contain at least one moiety chosen from formula (III):

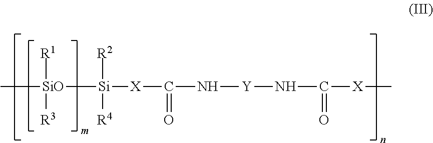

and formula (IV)

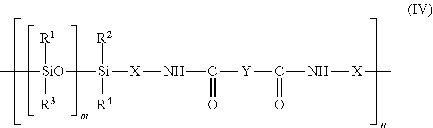

in which:
(a) R¹, R², R³ and R⁴ are the same or different and may be selected from the group consisting of methyl, ethyl, propyl, isopropyl, a siloxane chain, and phenyl;
(b) X is a linear or branched chain alkylene having 1-30 carbons;
(c) Y is selected from the group consisting of linear or branched chain alkylenes having 1-40 carbons;
(d) m is a number between 1 and 700;
(e) n is a number between 1 and 500.

Particularly preferred polyorganosiloxane-containing polymers useful herein are commercially available from Dow Corning under the tradenames DC 8178@ and DC 8179@, which are known under the INCI denomination of Nylon-611/Dimethicone Copolymer.

The at least one polyorganosiloxane-containing polymer is generally present in the cosmetic composition of the present invention in an amount ranging from about 1% to about 25% such as 1%, 2% 4% or 7% to about 10%, 20% or 25%, all weights being based on the weight of the composition as a whole.

### Silicone Elastomer

In a preferred embodiment, the composition comprises a silicone elastomer and, in particular, a non-emulsifying silicon elastomer. The non-emulsifying silicon elastomer may be in the form of a gel or a powder.

The “organopolysiloxane elastomer” or “silicon elastomer” or “silicone crosspolymer” thickens the composition, adds the cushiony (spongy) effect and to improves the application of the finished product. Also, it provides a very soft feel and mattifying effect after the application, which is especially advantageous for skin products.

The term “non-emulsifying” defines organopolysiloxane elastomers that do not contain in any hydrophilic chains, and in particular polyoxyalkylene (especially polyoxyethylene or polyoxypropylene) or polyglyceryl units. Thus, according to one particular embodiment of the invention, the composition comprises an organopolysiloxane elastomer that is free of polyoxyalkylene units and polyglyceryl units.

The non-emulsifying elastomers are described in U.S. Pat. No. 8,637,057, the disclosure of which is hereby incorporated by reference.

Silicone elastomers particularly useful in the present invention generally do not qualify as “silicone film-formers,” as they tend to form soft particulates that do not themselves form films.

One class of non-emulsifying silicone elastomers that are particularly useful in the present invention include vinyl modified silicone elastomers such as vinyl dimethicone crosspolymers, such as those having INCI designation DIMETHICONE CROSSPOLYMER, commercially available from Dow as DOWSIL EL-9240 SILICONE ELASTOMER BLEND or DIMETHICONE/VINYL DIMETHICONE CROSSPOLYMER, such as commercially available from Dow as DOWSIL 3903 LIQUID SATIN BLEND or from Shin-Etsu as USG-107A.

In an embodiment, the organopolysiloxane elastomer particles are conveyed in the form of a gel formed from an elastomeric organopolysiloxane included in at least one hydrocarbon-based oil and/or one silicone oil. In these gels, the organopolysiloxane particles are often non-spherical particles.

Not limited examples of silicone elastomers useful in this invention are dimethicone crosspolymer gels (available as blends of dimethicone crosspolymers in solvents) having viscosity values from about 150 and to about 700 mm²/s, from about 200 to about 650 mm²/s and from about 300 to about 600 mm²/s.

Particularly useful for this invention may be blends of high molecular weight silicone elastomers in volatile solvents, such as silicone oils, hydrocarbon oils and mixtures thereof, as per definition disclosed far along.

The specific but not limiting examples of silicone elastomeric gels applicable in this invention are represented by DC EL-8040 ID (INCI name: Isododecane (and) Dimethicone Crosspolymer) and DC EL-9140 DM (INCI name: Dimethicone (and) Dimethicone Crosspolymer) and DOWSIL EL-9240 Silicone Elastomer Blend (INCI name: Dimethicone (and) Dimethicone Crosspolymer), supplied by Dow Corning.

Non-limiting examples of silicone elastomers and their synthesis are disclosed, for example in U.S. Pat. No. 8,637,057 and US/20150174048, all of which are herein incorporated by reference.

The total amount of the at least one silicone elastomer is generally present in the cosmetic composition of the present invention in an amount ranging from about 1% to about 25% such as 1%, 2% or 4% to about 5%, 10%, 15% 20% or 25%, all weights being based on the weight of the composition as a whole.

### Platy Mineral

The inventors have found that use of a platy mineral is particularly useful in the present invention. By “platy mineral,” it is meant a particulate that is a mineral that develops crystals forms or faces of thin leaf-like layers (i.e., split along cleavage planes) and forms ‘sheet-like’ particulates having high aspect ratio. “Platy” minerals may be characterized by an aspect ratio and (average particle size/thickness) of at least about 5, 10 or 20 to about 100 or 200. As known to those skilled in the art, aspect ratio may be determined using, for example, shadowing, SEM, scanning white light interference microscopy, and the like.

The platy minerals useful in the present invention may have an average particle size that is from about 1 or 3 micrometers (m) to about 100 m or 150 m and may have 95% of particles in a range from about 0.5 m or 1 m to about 50 m, 75 m or 100 m. The platy mineral may have a thickness of about 0.1 m to 10 m. Average particle size may be determined using, for example, sieve analysis/screening; jet sieve, laser diffraction, light or electron microscopy, and the like. “Equivalent spherical” analysis may be used, and results may be reported using, for example, mean (in certain embodiments) or mode average (in other embodiments).

The platy minerals useful in the present invention may in certain embodiments have an oil absorption capacity, measured at the wet point, of at least 70 ml/100 g and a size expressed as volume-average diameter of at least 15 microns.

Examples of suitable platy minerals include coated or uncoated variants of layered silicates such as natural mica, synthetic mica (synthetic fluorophlogopite). Platy variants of certain other minerals may also be suitable, including certain other layered silicate clays such as bentonite clays, kaolin clays, as well as platy variants of certain materials falling outside the classification of layered silicates such as platy aluminum oxide, platy silicon dioxide, and certain glass flakes.

Natural and synthetic mica especially natural mica is particularly notable for the present invention. Accordingly in certain embodiments, the platy mineral comprises or consists entirely of natural or synthetic mica.

Mica is a layered silicate and specifically a highly platy phyllosilicate of aluminum and potassium and may be, for example of a muscovite or phlogopite variety. One suitable mica is an uncoated mica, available as MEARLMICA DD from BASF, having a bulk density of about 12 g/100 cc, and 95% of particles between 2 and 48 microns.

Coatings may be formed on the platy mineral may include those commonly used to provide optical effects (e.g., in effect pigments) or to provide tactile and/or stability benefits. Suitable coatings may include metal oxides such as titanium dioxides, iron oxides, bismuth oxides, silicone oxides, organic colorants (e.g., dyes) and combinations thereof. These may be formed using any number of known techniques such as precipitation via metal hydroxide and calcining; chemical vapor deposition, and the like. Other suitable coatings include various silicones, hydrocarbons, glutamates, lauroyl lysine, etc. According to certain embodiments, the platy mineral is uncoated.

According to certain notable embodiments, the at least one platy mineral is selected from the group consisting of a natural mica, a synthetic mica, and combinations thereof, and the platy mineral is present in a concentration in the composition that ranges from about 6.5%, 7% or 7.5% by weight to about 9% or 10% by weight, such as, in particular from about 6.5% by weight to about 10% by weight.

### Oil System

The compositions of the invention generally include an oil system that comprises, consist of, or consists essentially of at least one nonvolatile silicone and at least one volatile oil. The oil system serves as a vehicle for the composition to aid in spreadability of the composition across a keratinous surface such as the lips. The oil system further solubilizes and or disperses various other ingredients in the composition.

The oil system may be present in an amount ranging from about 40%, 50%, 60%, 65%, or 70% to about 70%, 80%, 85%, or 90%, all weights being based on the weight of the composition as a whole.

According to certain embodiments “oil,” refers to compounds having a melting point of less than about 30 C and generally insoluble in water and including a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties. In certain other embodiments, “oil” is meant to exclude surface active materials, such as those capable of depressing surface tension of water at 25 degrees C. to values less than about 50 dynes/cm when present in concentrations of 0.5% or less in deionized water.

In certain embodiments, the expression “volatile oil” means a non-aqueous compound generally insoluble in water and capable of evaporating on contact with the skin or the lips in less than one hour at room temperature and atmospheric pressure. In certain other embodiments “volatile oil” means any non-aqueous compound having a flash point of less than about 120° C., such as less than about 100° C., such as from about 40° C. to about 100° C.

Examples of suitable volatile oils include volatile hydrocarbon-based oils such as, for example, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, alcohols, and their mixtures.

Examples of volatile hydrocarbon-based oils include but are not limited to those given in Table 1 below.

TABLE 1

| Compound | Flash Point (° C.) |
|---|---|
| Isododecane | 43 |
| Isohexadecane | 102 |
| Isodecyl neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatile solvent may also be chosen from or include volatile silicone oils, which may be linear or cyclic, having a viscosity, at room temperature, of less than or equal to 6 cSt, and having from 2 to 7 silicon atoms, optionally substituted with alkyl or alkoxy groups of 1 to 10 carbon atoms.

Examples of suitable volatile silicone oils include, but are not limited to, those listed in Table 2 below.

TABLE 2

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or | | |
| D4) | | |
| Dodecamethylcyclohexasiloxane | 93 | 7 |
| (D6) | | |
| Decamethyltetrasiloxane(L4) | 63 | 1.7 |
| KF-96 A from Shin Etsu | 94 | 6 |
| PDMS (polydimethylsiloxane) | 56 | 1.5 |
| DC 200 (1.5 cSt) from Dow | | |
| Corning | | |
| PDMS DC 200 (2 cSt) from Dow | 87 | 2 |
| Corning | | |
| PDMS DC 200 (5 cSt) from Dow | 134 | 5 |
| Corning | | |
| PDMS DC 200 (3 St) from Dow | 102 | 3 |
| Corning | | |

The at least one volatile oil is generally present in the cosmetic composition of the present invention in an amount ranging from about 0.5%, 1%, 2%, 3% or 4% to about 4%, 5%, 6%, 8%, or 10% by weight based on the total weight of the composition, including all ranges and subranges within these ranges.

### Non-Volatile Silicone Oil

The oil system further includes at least one non-volatile silicone oil. The non-volatile silicone oil may be characterized as a non-aqueous compound generally fluid at room temperature, insoluble in water and including a plurality of Si—O bonds and a plurality of Si—C bonds. In certain other embodiments the non-volatile silicone oil has a flash point of greater than about 120° C., such as greater than about 150° C., such as greater than about 200° C.

The volatility of the oils can be determined using the evaporation speed as set forth in U.S. Pat. No. 6,338,839, the content of which is herein incorporated by reference.

Non-volatile silicone oils include low viscosity oils (having a viscosity from about 5 to about 10 centipoise) and high viscosity oils (having a viscosity of from about 100 to about 10,000 centipoise), and mixtures thereof. In contrast to waxes, oils are liquids at room temperature.

According to a certain embodiment of the present invention, the at least one silicone oil is a high viscosity oil which is a silicone oil. “High viscosity” means an oil having a viscosity greater than 100 cSt, particularly greater than 250 cSt at 25° C. Most particularly, the non-volatile oil is selected from a silicone oil. Such oils are described, for example in US 2011/0293550 and US 2004/0126350, both of which are herein incorporated by reference. In certain embodiments, the at least one silicone oil includes a plurality of silicone oils having varying individual viscosities. A weighted average viscosity, defined as the aggregated sum of each individual silicone oil's viscosity multiplied (weighted) by its relative weight fraction among all silicone oils may range from about 50 cps to about 100 cps.

Non-limiting examples of suitable non-volatile silicone oils include polymethylsiloxanes (PDMS) with a linear or cyclic silicone chain, which are liquid or pasty at room temperature, especially cyclopolydimethylsiloxanes (cyclomethicones) such as cyclohexasiloxane; polydimethyl-siloxanes (CTFA designation “dimethicones”) comprising alkyl or alkoxy groups, which are pendent or at the end of a silicone chain, these groups containing from 2 to 24 carbon atoms; polydiethyl siloxanes; and dimethicone fluids such as dimethicone fluids having a viscosity in particular ranges of centipoise (cps) or centistokes (cSt) when measured at 25° C.

Specific examples of suitable for this invention high viscosity silicone oils include, but are not limited to, Xiameter® silicone fluids from Dow Corning.

According to certain embodiments, the composition is substantially free or devoid of non-volatile solvents having at least one or more phenyl groups. These solvents are for example described in U.S. Pat. No. 8,945,525, the entire content of which is hereby incorporated by the reference.

The at least one non-volatile silicone oil may be present in the compositions of the present invention in an amount ranging from about 2% to about 75% by weight, such as from about 10%, 15%, 20% or 25% to about 40%, 50%, 60% or 75%, based on the total weight of the composition, including all ranges and subranges within these ranges.

### Ratios

According to certain embodiments, to provide surprisingly good drying time as well as tactile comfort, the at least one nonvolatile silicone oil and the at least one volatile oil are present in a particular weight ratio of nonvolatile silicone oil to volatile oil. The inventors have found this ratio should desirably be from about 4:1, 4.5:1 5:1 5.5:1 6:1 or 6.5:1 to about 7:1, 8:1, 9:1 or 10:1. This ratio is calculated by dividing the concentration by weight of the at least one nonvolatile silicone oil by the concentration by weight of the at least one volatile oil (for example, 10% total nonvolatile silicone oil(s) and 2% total volatile oil(s) would be a ratio of 5:1).

Furthermore in certain other embodiments, in order to provide aesthetic optical blurring in conjunction with mattity as well as the properties listed in the paragraph above in such compositions, it is desirable to have the at least one platy mineral is present in a certain weight ratio of platy mineral to total oil system (where oil system again refers to the total concentrations of nonvolatile silicone oils, volatile oils, and other oils). The inventors have found this ratio should desirably be from about 1:10, 1:9, or 1:8 to about 1:7, 1:6 or 1:5. This ratio is calculated, for example by dividing the concentration by weight of the oils by concentration by weight of the at least one platy mineral (for example, 10% oils and 2% platy minerals would be a ratio of 1:5).

### Colorants

The cosmetic compositions of the present invention may also contain at least one cosmetically acceptable colorant such as a pigment or dyestuff. Examples of suitable colorant include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface treated within the scope of the present invention but are not limited to treatments such as silicones, perfluorinated compounds, lecithin, and amino acids.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

The at least one colorant may be present in the compositions of the present invention in an amount ranging from about 0.5%, 0.6% or 1% to about 2%, 3.5%, 5% or 10% based on the total weight of the composition, including all ranges and subranges within these ranges.

### Wax

The cosmetic compositions of the present invention optionally may contain at least one wax.

For the purposes of the present invention, a wax is a lipophilic fatty compound that is solid at room temperature (25° C.), has a reversible solid/liquid change of state (that is, the state of the material may change based on temperature), has a melting point greater than 45° C., preferably greater than 55° C., more preferably between about 65° C. to about 120° C., and has anisotropic crystal organization in the solid state. The melting point of the wax may be measured using a differential scanning calorimeter (DSC), for example the calorimeter sold under the name DSC 30 by Mettler. For waxes that are derived from petroleum, such as microcrystalline wax, the melting point may be measured according to the drop ASTM method, D-127.

The waxes are those generally used in cosmetics and dermatology. The waxes may be of natural origin, for instance beeswax, carnauba wax, candelilla wax, ouricoury wax, Japan wax, cork fiber wax or sugar cane wax, paraffin wax, lignite wax, microcrystalline waxes, lanolin wax, montan wax, ozokerites and hydrogenated oils, for instance hydrogenated jojoba oil.

The waxes also may be of synthetic origin, for instance polyethylene waxes derived from the polymerization of ethylene, waxes obtained by Fischer-Tropsch synthesis, esters of fatty acids and of glycerides that are solid at 40° C.

Particular waxes include polyethylene waxes, for example the product sold under the name Performance 500-L Polyethylene (New Phase Technology), and polymethylene waxes, for instance the product sold under the name Cirebelle 303 (Sasol).

The cosmetic compositions of the present invention may contain at least one polypropylsilsesquioxane wax substituted with alkyl units having at least 30 carbons.

Polypropylsilsesquioxane waxes, in general, have been disclosed in patent publication WO2005/100444 and U.S. Pat. No. 8,586,013, the entire contents of which are hereby incorporated by reference.

A particularly preferred polypropylsilsesquioxane wax for use in the present invention is a C30-45 ALKYLDIMETHYLSILYL POLYPROPYLSILSESQUIOXANE commercially available from DOW CORNING under the tradename SW-8005 C30 Resin Wax.

When present in the instant compositions, the at least one wax may be present in an amount ranging from about 0.01% to about 5% by weight, typically from about 0.03% or 0.5% to 1%, 2%, 3% or 4% by weight. According to certain embodiments the compositions have less than about 4% of total waxes. According to certain other embodiments, the compositions are substantially free of hydrocarbon waxes.

### Fillers

Fillers that may be used in the compositions of the invention include those that provide a bulking, opacifying or especially, a mattifying effect and are not encompassed by the above definition for “platy mineral” Examples include certain minerals having various chemistries such as silica powder; talc; polyamide particles; polyethylene powders; microspheres based on acrylic copolymers, such as those based on ethylene glycol dimethacrylate/lauryl methacrylate copolymer; expanded powders such as hollow microspheres; powders of natural organic materials such as crosslinked or noncrosslinked corn starch, wheat starch or rice starch, such as the powders of starch crosslinked with octenyl succinate anhydride; silicone resin microbeads; and certain clays (bentone, laponite, saponite, etc.); and mixtures thereof.

Particularly useful fillers for the present invention are mattifying agents/fillers. Particularly suitable mattifying agents are those having a specific surface are (measured for example by BET) that is at least about 100 m²/g. Suitable examples include aerogels such as certain microcrystalline celluloses and hydrophobic silica aerogels. Silica aerogels are particularly notable.

Silica aerogels are porous materials obtained by replacing (by drying) the liquid component of a silica gel with air. They are generally synthesized via a sol-gel process in liquid medium and then dried, usually by extraction of a supercritical fluid, the one most commonly used being supercritical CO₂. This type of drying makes it possible to avoid shrinkage of the pores and of the material. The sol-gel process and the various drying processes are described in detail in Brinker CJ., and Scherer G. W., Sol-Gel Science: New York: Academic Press, 1990. Silica aerogels, in general, have been disclosed in U.S. Pat. No. 9,320,689, the entire content of which is hereby incorporated by reference.

As hydrophobic silica aerogels that may be used in the invention, examples that may be mentioned include the aerogel sold under the name VM-2260 (INCI name: Silica silylate), by the company Dow Corning, the particles of which have a mean size of about 1000 microns and a specific surface area per unit of mass ranging from 600 to 800 m²/g.

Mention may also be made of the aerogels sold by the company Cabot under the references AEROGEL TLD 201, AEROGEL OGD 201, AEROGEL TLD 203, ENOVA® AEROGEL MT 1100, ENOVA AEROGEL MT 1200.

One example is the aerogel sold under the name VM-2270 (INCI name: Silica silylate), by the company Dow Corning, the particles of which have a mean size ranging from 5-15 microns and a specific surface area per unit of mass ranging from 600 to 800 m²/g.

The (mattifying) fillers may be present in the compositions of the present invention in an amount ranging from about 1% to about 10% by weight, such as from about 0.1%, 0.5%, or 1% by weight to about 2%, 3%, 5% or 10%, based on the total weight of the composition, including all ranges and subranges within these ranges.

In particular, silica aerogel particles if used can be used in the inventive compositions from 0.1% to about 6% by weight, more preferably from about 0.2%, 0.3%. or 0.5% or 0.6% to about 0.6%, 1%. 1.5% 2% or 5% by weight.

In certain embodiments, if expanded perlite is included among the fillers, the concentration by weight is less than 10%, such as less than 5%, such as less than 1%. In certain other embodiments, the composition is substantially free of swellable clays (clay materials that is capable of swelling in water). An example of a swellable clay is smectite clays. The crystal structure of the smectite group, is an octahedral alumina sheet between two tetrahedral silica sheets. In one notable embodiment, the swellable clay is bentonite. Bentonite is a rock formed of highly colloidal and plastic clays composed mainly of montmorillonite, a clay mineral of the smectite group, and is produced by in situ devitrification of volcanic ash. In addition to montmorillonite, bentonite may contain feldspar, cristobalite, and crystalline quartz. Bentonite has an ability to form thixotrophic gels with water, an ability to absorb large quantities of water. Variations in interstitial water and exchangeable cations in the interlayer space affect the properties of bentonite and thus the commercial uses of the different types of bentonite.

### Additives

The compositions of the present invention may further comprise at least one cosmetically or dermatologically acceptable additive such as an additional thickener, a plasticizer, an antioxidant, an essential oil, a botanical extract, a fragrance, a preserving agent, a fragrance, a pasty fatty substance, a neutralizing agent, and a polymer, and cosmetically active agents and/or dermatological active agents such as, for example, oils (other than those described above), moisturizers, vitamins, essential fatty acids and medicaments.

As per this invention, the additives are incorporated from about 0.01%, 0.5% or 1% to about 1%, 2% or 5% by weight.

In certain embodiments, the cosmetic composition useful for application to the lips is a liquid lip cosmetic composition that includes (1) from about 40% by weight to about 80% by weight an oil system that comprises or consists of at least one nonvolatile silicone oil and at least one volatile oil; (2) at least one silicone film-former; (3) at least one silicone elastomer; (4) from about 6.5% to about 10% mica; and (5) silica aerogel (such as from about 0.6% to about 1.5% silica aerogel). The at least one nonvolatile silicone oil and the at least one volatile oil are present in a weight ratio of nonvolatile silicone oil to volatile oil from about 4:1 to about 10:1.

The compositions of the present invention are useful as compositions for making up the skin, in particular the lips. The compositions may be used for example as single layer treatment or as a basecoat of top coat.

Compositions may be made by methods known to those skilled in the art, such as by charging a vessel with one or more solvents or oil and adding various ingredients and mixing. Pigments may be pre-ground into a suspension or slurry prior to adding.

The present invention will be better understood from the examples which follow. The examples are intended to be nonrestrictive and explanatory only, with the scope of the invention defined by the claims.

### EXAMPLES

The present invention will be better understood from the examples which follow. The examples are intended to be nonrestrictive and explanatory only, with the scope of the invention defined by the claims.

The following compositions, Inventive Examples E1 and E2 (with different colorant concentrations) and Comparative Examples C1-C11 were prepared as per Table 1, Table 2 and Table 3.

Compositions were generally prepared using by mixing of pigment, isododecane silicone resin and grinding to create a pigment paste. The blend is processed using Disconti Mill until the paste is passed the Hegman Gauge test (ASTM D1210-05). Then, the paste grind is added to the remaining ingredients. The mixture is heated to about 80° C. and stirred, until a homogeneous liquid composition is obtained. After that, the compositions are cooled to room temperature and transferred to desired containers and/or applicators. “Other silicones*” in the tables refer to silicone resin, silicone elastomer, silicone wax, and polyorganosiloxane copolymer. “Other” ingredients refer to preserving agents and other additives as described above.

Compositions were evaluated by having a user apply the composition to her lips using a conventional liquid lipstick applicator. Degree of blurring and mattity were visually evaluated applied to the lips and visually and tactilely evaluated for blurring, mattity, tactile comfort, wear. Dry time was evaluated by performing a kiss test on the back of the hand. Unless otherwise noted below, the samples were acceptable according to each of these attributes.

Compositions without volatile solvent (here, isododecane) have an inferior drying time (see C11), yet if one attempts to add increasing volatile oil to where the ratio of nonvolatile silicone oil (here, dimethicone) to volatile oil (here isododecane) is below about 4:1, the user starts to experience tactile discomfort such as a feeling of dryness or tightness (see C1-C10). Using low amounts of volatile oil, however also require increasing proportions of other ingredients. The inventors have found that using platy mineral (mica, in the example below), compared with other fillers and pigments is very useful in this regard. For example, the inventors have found that samples where the ratio of platy pigment (here, mica) to oil system (here, dimethicone plus isododecane) is from about 1:10 to about 1:5 have surprisingly good performance.

Further, since mattity is also desirable, and platy pigments are not typically sufficient to provide enough mattity, and additional mattifying agent (see use of silica silylate below) is needed.

TABLE 1

| Ingredient | E1-E2 | C1 | C2 | C3 | C4 |
|---|---|---|---|---|---|
| Dimethicone | 46% | 40% | 31% | 40% | 40% |
| Isododecane | 6.3% | 12% | 33% | 14% | 12% |
| Other Silicones* | 35% | 35% | 20% | 35% | 25% |
| Mica | 8.5% | — | — | — | 8.0% |
| Silica silylate | 1.0% | 0.50% | 0.75% | — | 0.50% |
| Spherical silica | — | — | 5.2% | — | — |
| Nylon-12 | — | — | — | — | — |
| Colorants | 1.0% or | 10% | 6.0% | 2.0% | 2.0% |
| | 1.5% | | | | |
| Other | Q.S. | Q.S. | Q.S. | Q.S. | Q.S. |
| dimethicone:isododecane | 7.3:1 | 3.3:1 | 0.84:1 | 2.8:1 | 3.3:1 |
| mica:(dimethicone + | 1:6.2 | 0 | 0 | 0 | 1:6.6 |
| isododecane) | | | | | |
| EVALUATION RESULTS | Excellent | Minimal | Some | Minimal | Excellent |
| | blurring, | blurring, | blurring, | blurring, | blurring, |
| | mattity, & | some tactile | some tactile | some tactile | some tactile |
| | tactile | discomfort; | discomfort; | discomfort; | discomfort; |
| | comfort | good | good | good | good |
| | | mattity, | mattity; | mattity; | mattity |
| | | excellent | | good wear | |
| | | wear | | | |

TABLE 2

| Ingredient | C5 | C6 | C7 | C8 | C9 |
|---|---|---|---|---|---|
| Dimethicone | 34% | 36% | 23% | 46% | 46% |
| Isododecane | 30% | 14% | 27% | 16% | 18% |
| Other Silicones* | 28% | 28% | 22% | 30% | 28% |
| Mica | — | 8.0% | — | — | 4% |
| Silica silylate | 0.76% | 0.6% | — | 1.0% | 1.0% |
| Perlite | — | — | 15% | — | — |
| Hollow spherical silica | — | — | — | 2.0% | 5.0% |
| Colorants | 5.0% | 3.0% | 8.8% | 2.0% | 1.0% |
| Other | Q.S. | Q.S. | Q.S. | Q.S. | Q.S. |
| dimethicone:isododecane | 1.1:1 | 3.3:1 | 0.84:1 | 2.9:1 | 2.5:1 |
| mica:(dimethicone + | 0 | 1:7.3 | 0 | 0 | 1:16 |
| isododecane) | | | | | |
| EVALUATION RESULTS | Minimal | Good | Some tactile | Minimal | Some |
| | blurring, | blurring, | discomfort; | blurring, | blurring, |
| | some tactile | some tactile | good | some | some |
| | discomfort; | discomfort; | mattity. | tactile | tactile |
| | good | good | | discomfort; | discomfort; |
| | mattity. | mattity. | | good | good |
| | | | | mattity. | mattity. |

TABLE 3

| Ingredient | C10 | C11 |
|---|---|---|
| Dimethicone | 46% | 52% |
| Isododecane | 18% | 0% |
| Other Silicones* | 26% | 35% |
| Mica | 4.0% | 8.5% |
| Silica silylate | 1.0% | 1.0% |
| Nylon-12 | 5.0% | — |
| Hollow spherical silica | | — |
| Colorants | 1.0% | 1.5% |
| Other | Q.S. | Q.S. |
| dimethicone:isododecane | 2.5:1 | N/A |
| mica:(dimethicone + | 1:16 | 1:6.1 |
| isododecane) | | |
| EVALUATION RESULTS | Minimal blurring, | Good blurring, some |
| | tactile discomfort; | tactile discomfort; good |
| | good mattity | mattity, poor dry time. |

The mixture of pigment, isododecane and MQ resin was ground to create a pigment paste. The blend was processed using a 3 Roller Mill until the paste passed the Hegman Gauge test (ASTM D1210-05). Then, the paste grind was added to the remaining ingredients. The mixture was heated to 80° C. and stirred, until a homogeneous liquid composition was obtained. After that, the inventive composition was cooled down to the room temperature and transferred to desired containers and/or applicators.

What is claimed is:

1. A method of making up the lips, comprising applying to the lips an anhydrous composition comprising:
at least one nonvolatile silicone oil;
at least one volatile oil;
at least one film-former;
at least one colorant;
at least one platy mineral; and
a mattifying agent; wherein the at least one nonvolatile silicone oil and the at least one volatile oil are present in a weight ratio of nonvolatile silicone oil to volatile oil from about 4:1 to about 10:1.

2. The method of claim 1, wherein the at least one nonvolatile silicone oil and the at least one volatile oil compose an oil system and wherein the at least one platy mineral is present in a weight ratio of platy mineral to oil system that is from about 1:10 to about 1:5.

3. The method of claim 1, wherein the at least one platy mineral is selected from a group consisting of a natural mica, a synthetic mica, and combinations thereof wherein the at least one platy mineral is present in a concentration in the composition that ranges from about 6.5% by weight to about 10% by weight.

4. The method of claim 1, wherein the at least one nonvolatile silicone oil and the at least one volatile oil are present in a total concentration in the composition that ranges from about 40% by weight to about 80% by weight.

5. The method of claim 1, wherein the at least one volatile oil is present in a concentration in the composition from about 4% by weight to about 10% by weight.

6. The method of claim 1, wherein the weight ratio of nonvolatile silicone oil to volatile oil in the composition is from about 4:1 to about 8:1.

7. The method of claim 1, wherein the at least one volatile oil comprises one or more C₈ to C₁₆ alkanes.

8. The method of claim 1, wherein the mattifying agent comprises a silica aerogel, and the silica aerogel is present in a concentration in the composition from about 0.6% by weight to about 2% by weight.

9. The method of claim 1, wherein the at least one colorant is present in a concentration in the composition from about 0.6% by weight to about 3.5% by weight.

10. The method of claim 1, wherein the composition is liquid.

11. The method of claim 1, wherein the composition comprises less than about 4% of wax.

12. The method of claim 1, wherein the composition further comprises a silicone elastomer.

13. The method of claim 1, wherein the at least one film-former comprises a siloxysilicate resin.

14. The method of claim 1, wherein the at least one volatile oil comprises one or more C₈ to C₁₆ alkanes, and wherein the composition is a liquid.

15. A liquid lip cosmetic composition comprising:
from about 40% by weight to about 80% by weight an oil system comprising at least one nonvolatile silicone oil and at least one volatile oil;
at least one silicone film-former;
at least one silicone elastomer;
from about 6.5% to about 10% mica; and
silica aerogel; wherein the at least one nonvolatile silicone oil and the at least one volatile oil are present in a weight ratio of nonvolatile silicone oil to volatile oil from about 4:1 to about 10:1.

16. The liquid lip composition of claim 1, wherein the at least one platy mineral is present in a weight ratio of platy mineral to oil system that is from about 1:10 to about 1:5.

17. The liquid lip composition of claim 1, wherein the at least one volatile oil comprises one or more C₈ to C₁₆ alkanes.

18. The liquid lip composition of claim 1, wherein the at least one volatile oil is present in a concentration that is from about 4% by weight to about 10% by weight of the composition.

19. The liquid lip composition of claim 1, wherein the composition comprises less than about 4% of wax.

20. The liquid lip composition of claim 1, wherein the silica aerogel is present in a concentration in the composition from about 0.6% by weight to about 2% by weight.
